(19) United States
(12) Patent Application Publication
(10) Pub. No.: US 2021/0253448 A1
(43) Pub. Date: Aug. 19, 2021

(54) ASSEMBLY FOR A DEVICE FOR DISINFECTING A FLUID, PRODUCTION METHOD, DISINFECTING DEVICE, AND DEVICE FOR DISPENSING A DISINFECTED FLUID AT A POINT OF CONSUMPTION

(71) Applicant: HYTECON AG, Luzern (CH)

(72) Inventor: Maximilian KLINK, Luzern (CH)

(73) Assignee: HYTECON AG, Luzern (CH)

(21) Appl. No.: 17/059,090

(22) PCT Filed: Aug. 26, 2019
(86) PCT No.: PCT/EP2019/072693

§ 371 Date: Nov. 25, 2020

(30) Foreign Application Priority Data
Aug. 31, 2018 (DE) 10 2018 121 327.1

(51) Int. Cl. C02F 1/32

(52) U.S. Cl. CPC C02F 1/325; C02F 2201/3228; C02F 2307/06; C02F 2201/002; C02F 2303/04

(57) ABSTRACT

An assembly of a device for disinfecting a fluid is presented. The assembly includes: a container; a reactor chamber, which is arranged in the container and is designed to receive a fluid to be disinfected, which enters the reactor chamber via an inlet of the reactor chamber and can exit the reactor chamber via an outlet of the reactor chamber; and an irradiating apparatus, which is designed to provide UV light beams and to radiate the UV light beams into the reactor chamber in order to disinfect the fluid in the reactor chamber, where a container wall of the container is composed of multiple layers and an inner layer of the container wall, which faces and surrounds the reactor chamber, is made of polytetrafluoroethylene and has a layer thickness of at least 1.0 mm.

The invention relates to an assembly for a device for disinfecting a fluid, to a production method, to a disinfecting device and to a device for dispensing a disinfected fluid at a point of consumption.

### BACKGROUND

The disinfection of fluids, such as water or drinking water, is provided for in conjunction with a wide variety of applications. A contaminated or unsuitable fluid is disinfected so that it can then be provided in a disinfected form for further use. For example, water is disinfected in this manner in order to provide it, for example, as drinking water, in particular at a point of consumption (point of use).

Document WO 2018/050 144 A1 discloses a decentralized water disinfecting device. UV rays from UV light-emitting diodes are used to disinfect the water in a reactor chamber. In document DE 10 2016 122 075 A1, an operating method and a device for monitoring a UV disinfection system are disclosed. UV rays are used to disinfect a fluid in a reactor chamber.

Document DE 100 37 289 A1 describes a molded body that has a barrier film that is back injection molded with a polymer material.

### SUMMARY

The object of the invention is to provide improved technologies in connection with systems or devices for disinfecting a fluid that allow more efficient disinfection of the fluid, in particular water.

To achieve this objective, an assembly for a device for disinfecting a fluid and a method for producing such an assembly according to independent claims 1 and 9 are created. Furthermore, a disinfecting device and a device for dispensing a disinfected fluid at a point of consumption according to independent claims 7 and 8 are provided. Embodiments are the subject matter of dependent subclaims.

According to one aspect, an assembly for a device for disinfecting a fluid is created that has a container and a reactor chamber that is arranged in the container. The reactor chamber is configured to accommodate a fluid to be disinfected, which enters the reactor chamber via an inlet thereof and may exit the reactor chamber via an outlet thereof. Furthermore, an irradiation device is provided that is configured to provide UV rays and to radiate them into the reactor chamber in order to disinfect the fluid in the reactor chamber. A container wall of the container has a multilayer design, an inner layer of the container wall, which faces and surrounds the reactor chamber, being made of polytetrafluoroethylene and having a layer thickness of at least 1.0 mm. The inner layer of the container wall is back injection molded with a plastic material. An outer layer of the container wall is formed with the back injection molded plastic material.

According to a further aspect, a method for producing an assembly for a device for disinfecting a fluid is created, the method comprising the following steps: producing a container having a reactor chamber that is formed in the container and configured to accommodate a fluid to be disinfected, which enters the reactor chamber via an inlet thereof and may exit the reactor chamber via an outlet thereof; and forming an irradiation device on the container, which irradiation device is arranged to provide UV rays and to radiate them onto the fluid in the reactor chamber in order to disinfect the fluid in the reactor chamber. A container wall of the container has a multilayer design, an inner layer of the container wall, which faces and surrounds the reactor chamber, being made of polytetrafluoroethylene and having a layer thickness of at least 1.0 mm. The inner layer of the container wall is back injection molded with a plastic material. An outer layer of the container wall is formed with the back injection molded plastic material.

Furthermore, a disinfecting device for disinfecting a fluid, i.e., a liquid or a gas, is provided with the assembly. For example, the disinfection of water or air may be provided.

According to a further aspect, a device for dispensing a disinfected fluid at a point of consumption is created, the device having a supply connection and a discharge connection. A fluid, for example water or air, to be dispensed at a point of consumption may be supplied via the supply connection. The fluid may be dispensed at the point of consumption via the discharge connection. A disinfecting device for disinfecting the fluid is provided, the disinfecting device being connected to the supply connection and to the discharge connection in such a way that the fluid may be disinfected in the disinfecting device after being supplied via the supply connection and before being dispensed at the point of consumption.

The proposed technology provides a container optimized for a disinfecting device, in which container the inner layer surrounding the reactor chamber is formed from the inert material polytetrafluoroethylene (PTFE) with a minimum layer thickness of 1.0 mm. This allows the light beams radiated to disinfect the fluid to be reflected by the inner layer to the greatest extent possible and to be sent back into the fluid to be disinfected, for example water, to thereby contribute multiple times to the disinfection. It was determined that the provided thickness of the inner layer largely prevents a loss of light beams due to transmission through the inner layer.

The inner layer may substantially completely surround the reactor chamber. Gaps or recesses may be limited to a small number of wall regions, in particular the region of the inlet and the outlet as well as, optionally, the region of an optical window through which the light beams may be coupled in if the lighting device is arranged outside the reactor chamber. Polytetrafluoroethylene forms an inert material such that an inert inner layer of the container wall is provided.

The lighting device may be formed inside and/or outside the reactor chamber.

In particular, disinfection of water or drinking water may be provided to purify water into drinking water or to improve the water quality of existing drinking water by means of disinfection.

It may be provided that a filtration device is arranged upstream of the reactor chamber, in which filtration device the fluid to be disinfected is filtered before being brought to the reactor chamber, for example by using an activated charcoal filter. The filtration device may be designed as part of the disinfecting device or to be upstream thereof.

The inner layer of the container wall is back injection molded with a plastic material. The inner layer and the layer that is produced by means of back injection molding and made of a plastic material, for example a polymer material, may have a substantially equal layer thickness or different layer thicknesses in the layered composite of the container wall. In this case, it may be provided that the inner layer has a smaller layer thickness than the plastics layer.

An outer container wall may be formed with back injection molded plastics material. The layer that is produced by means of back injection molding and made of the plastic material forms the outer layer of the container wall here. Further layers may optionally be provided for the construction of the container wall.

In the case of the inner layer of polytetrafluoroethylene, an inner surface facing the reactor chamber may be designed to reflect the UV rays diffusely. In one embodiment, the inner surface of the inner layer is arranged to diffusely reflect UV light within a range of about 200 nm to about 300 nm.

The inner layer may have a degree of reflection of at least about 90% for the light beams. Alternatively, the inner layer may have a degree of reflection of at least about 95% or at least about 98% for the light beams. In one embodiment, the degree of reflection for the light beams is no more than about 98%. The degree of reflection may, in particular, be designed in the indicated size for light beams in the UV range. In one embodiment, the inner layer has the indicated degree of reflection for light beams within a range of about 250 nm to about 1000 nm, alternatively within a range of about 250 nm to about 2500 nm.

The inner layer is UV-resistant. The UV resistance may, in particular, mean that the inner layer does not become discolored and/or brittle due to irradiation with UV light.

The inner layer may be thermally resistant up to temperatures of about 200° C. In one embodiment, it may be provided that the inner layer is thermally resistant up to a temperature of about 260° C.

The inner layer may have a layer thickness of at least 1.2 mm, alternatively of at least 1.5 mm. Larger layer thicknesses may support the desired visual properties of the inner layer with respect to avoiding losses of the light beams incident thereon.

The lighting device may at least in part be arranged in the reactor chamber, and a portion of the lighting device arranged in the reactor chamber may at least in some portions have an outer polytetrafluoroethylene (PTFE) coating. The portion of the lighting device may, for example, be provided with a PTFE film. The lighting device may have a printed circuit board or card on which one or more light sources for generating the UV rays are arranged. The surface of the printed circuit board may be provided in full or in part with the outer PTFE coating, it being possible to provide punched-out portions or openings for light sources. The outer PTFE coating may have one or more of the properties described above as described for the inner layer of the reactor chamber.

The irradiation device has a light source that is arranged to provide the UV rays for disinfecting the fluid. One or more light-emitting diodes may be provided as a light source for the UV rays or light beams in a different spectral range.

In conjunction with the disinfecting device for disinfecting a fluid, for example water or air, and the device for dispensing the disinfected fluid at the point of consumption, one or more of the embodiments described above may be provided accordingly.

The above explanations regarding embodiments apply mutatis mutandis in connection with the method for producing the assembly for a device for disinfecting a fluid.

### DESCRIPTION OF EMBODIMENTS

Further embodiments will be explained in more detail below with reference to the drawings, in which:

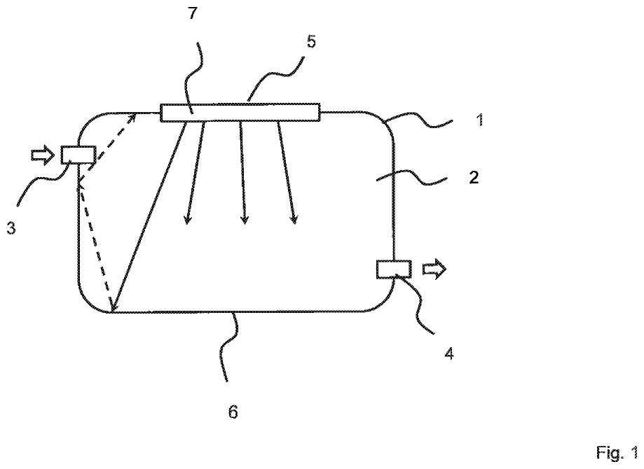

FIG. 1 is a schematic representation of an assembly for a device for disinfecting a fluid, in particular water, having a container and an irradiation device;

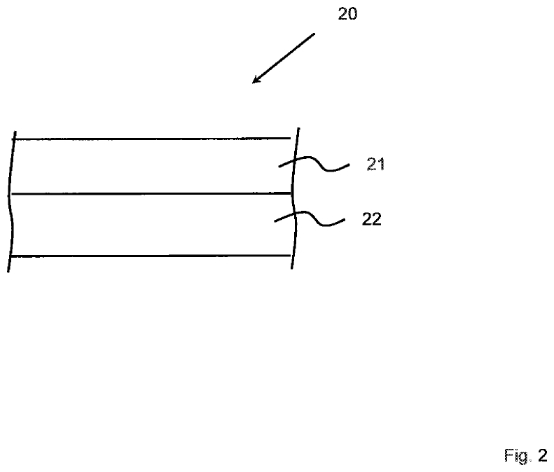

FIG. 2 is a schematic representation of a portion of a container wall; and

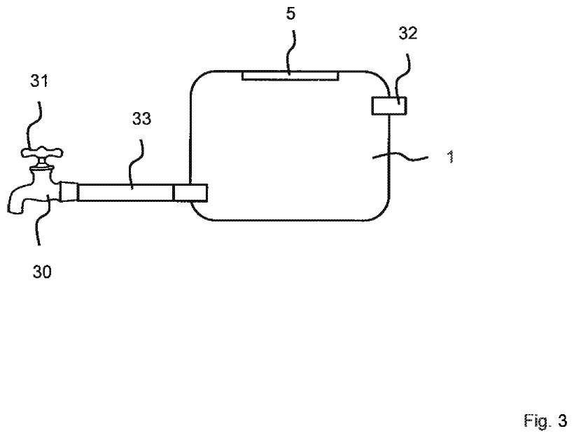

FIG. 3 is a schematic representation of a device for dispensing a disinfected fluid at a point of consumption having the assembly from FIG. 1.

FIG. 1 is a schematic representation of an assembly for a device for disinfecting a fluid, in particular water or drinking water or a gas such as air. A container 1 is provided in which a reactor chamber 2 is provided to accommodate a fluid to be disinfected. For this purpose, the fluid to be disinfected flows into the reactor chamber via an inlet 3 and exits said reactor chamber via an outlet 4.

An irradiation device 5 is arranged on the container, which irradiation device is accommodated in a container wall 6 in the illustrated embodiment. The irradiation device 5 has a light source 7 for generating UV rays in order to radiate them onto the fluid in the reactor chamber 2 to disinfect the fluid. The radiated UV rays are then reflected diffusely on an inner surface 8 of the container wall.

According to the wall portion 20 illustrated in an enlarged manner in FIG. 2, the container wall 6 is of a multilayer construction having an inner layer 21 and an outer layer 22. The inner layer 21 consists of polytetrafluoroethylene (PTFE). In this case, so-called optical PTFE may be used, which has preferred properties for the diffuse reflection of the light beams, in particular a high degree of reflection for the light beams, for example a degree of reflection between about 90% and about 98%. Such a degree of reflection may be provided, for example, for the UV rays.

The inner layer 21 made of PTFE is back injection molded with a plastic material to produce the outer layer 22. For this purpose, a PTFE film with the desired layer thickness is placed into an injection molding tool (not shown) and back injection molded with the plastic material during the production of the container 1. Injection molding tools and methods for back injection molding a material are known per se in various embodiments.

FIG. 3 is a schematic representation of a device for dispensing a disinfected fluid, whether it be a liquid or a gas, at a point of consumption 30, which device is equipped, for example, with a water faucet 31. The fluid to be dispensed and disinfected is supplied via a supply connection 32 to enter the container 1 for disinfection. The outlet of the container 1 is coupled to a discharge connection 33, which leads to the point of consumption 30.

The features disclosed in the above description, the claims and the drawings may be significant to the implementation of the various embodiments both individually and in any combination.

1. An assembly for a device for disinfecting a fluid, comprising:
a container;
a reactor chamber that is arranged in the container and is configured to accommodate a fluid to be disinfected, which enters the reactor chamber via an inlet thereof and may exit the reactor chamber via an outlet thereof; and
an irradiation device that is configured to provide UV rays and to radiate them into the reactor chamber in order to disinfect the fluid in the reactor chamber;
wherein a container wall of the container has a multilayer design and an inner layer of the container wall, which faces and surrounds the reactor chamber is made of polytetrafluoroethylene and has a layer thickness of at least 1.0 mm.

2. The device according to claim 1, wherein the inner layer of the container wall is back injection molded with a plastic material.

3. The device according to claim 2, wherein an outer layer of the container wall is formed with the back injection molded plastic material.

4. The device according to claim 1, wherein an inner surface facing the reactor chamber is configured to diffusely reflect the UV rays in the case of the inner layer of polytetrafluoroethylene.

5. The device according to claim 1, wherein the inner layer has a degree of reflection of at least about 90% for the UV rays.

6. The device according to claim 1, wherein the inner layer is thermally resistant up to temperatures of about 200° C.

7. The device according to claim 1, wherein the inner layer has a layer thickness of at least 1.2 mm, alternatively of at least 1.5 mm.

8. The device according to claim 1, wherein the irradiation device is at least in part arranged in the reactor chamber and in that a portion of the irradiation device arranged in the reactor chamber has, at least in some portions, an outer polytetrafluoroethylene coating.

9. A disinfecting device for disinfecting a fluid, comprising an assembly according to claim 1.

10. A device for dispensing a disinfected fluid at a point of consumption, comprising
a supply connection via which a fluid to be dispensed at a point of consumption may be supplied;
a discharge connection which the fluid may be dispensed at the point of consumption; and
a disinfecting device for disinfecting the fluid according to claim 9, wherein the disinfecting device is connected to the supply connection and to the discharge connection in such a way that the fluid may be disinfected in the disinfecting device after being supplied via the supply connection and before being dispensed at the point of consumption.

11. A method for producing an assembly for a device for disinfecting a fluid, comprising:
producing a container having a reactor chamber that is formed in the container and is configured to accommodate a fluid to be disinfected, which enters the reactor chamber via an inlet thereof and may exit the reactor chamber via an outlet thereof; and
forming an irradiation device on the container, which irradiation device is configured to provide UV rays and to radiate them onto the fluid in the reactor chamber to disinfect the fluid in the reactor chamber;
wherein a container wall of the container has a multilayer design and an inner layer of the container wall, which faces and surrounds the reactor chamber, is made of polytetrafluoroethylene and has a layer thickness of at least 1.0 mm.
